(19) United States
(12) Patent Application Publication
(10) Pub. No.: US 2008/0194658 A1
(43) Pub. Date: Aug. 14, 2008

(54) Sterol Carrier Protein-2 Inhibitors for Lowering Cholesterol and Triglyceride Levels in Mammals

(76) Inventors: Que Lan, Madison, WI (US); Jeff Lorch, Middleton, WI (US)

Correspondence Address: QUARLES & BRADY LLP, 33 E. MAIN ST, SUITE 900, P.O. BOX 2113, MADISON, WI, 53701-2113, US

(21) Appl. No.: 11/953,956

(22) Filed: Dec. 11, 2007

Related U.S. Application Data
(60) Provisional application No. 60/874,167, filed on Dec. 11, 2006.

(51) Int. Cl. A61K 31/426; A61P 43/00

(52) U.S. Cl. 514/370

(57) ABSTRACT

Methods of treating high serum levels of total cholesterol, low density lipoprotein and triglycerides in a mammal by administering therapeutically effective doses of N-(4-{[4-(3,4-dichlorophenyl)-1,3-thiazol-2-yl]amino}phenyl)acetamide or a salt thereof such as a hydrochloride or hydrobromide salt.

### CROSS-REFERENCE TO RELATED APPLICATIONS

This application claims the benefit of U.S. Provisional Application No. 60/874,167 filed Dec. 11, 2006, which is incorporated herein by reference in its entirety.

### GOVERNMENT FUNDING STATEMENT

This invention was made with United States government support awarded by the following agencies: ARMY/SMDC W9113M-05-1-006 and USDA/CSREES Grants 05-CRHF-0-6055 and 08-CRHF-0-6055. The United States has certain rights in the invention.

### FIELD OF THE INVENTION

The field of the invention concerns chemical compounds useful for lowering cholesterol and triglyceride serum levels in mammals by binding to conserved, intracellular, sterol carrier protein-2 molecules, which are primarily expressed in the liver and intestine.

### BACKGROUND OF THE INVENTION

Sterol carrier protein-2 (SCP-2) is a conserved intracellular sterol carrier protein. (Gallegos A M et al., Gene structure, intracellular localization, and functional roles of sterol carrier protein-2, Prog Lipid Res 2001 40(6):498-563). SCP-2 was reported in early literature as related to delivery of cholesterol from preformed stores to and into mitochondria for initiation of steroid hormone synthesis. (Chanderbhan R et al., Sterol Carrier Protein₂: Delivery of cholesterol from adrenal lipid droplets to michchondria for pregnenolone synthesis, J Bio Chem 1982 257(15):8928-8934). SCP-2 binds lipids in both vertebrate and insect systems, whereby its affinity for cholesterol is much greater than its affinity for fatty acids. In vertebrates, expression of SCP-2 is most prominent in the liver and intestine. In invertebrates, SCP-2 is detected mostly in the gut tissue. (Krebs K C et al., Isolation and Expression of a Sterol Carrier Protein-2 Gene from the Yellow Fever Mosquito, Aedes aegypti. Insect Mol Biol 2003 12(1):51-60 and Lan Q et al., Subcellular localization of mosquito sterol carrier protein-2 and sterol carrier protein-x, J Lipid Res 2004 45(8):1468-1474). While studying the mosquito sterol carrier protein (AeSCP-2), it has been reported that there exists conversed and divergent functions between vertebrate and invertebrate SCP-2. (Krebs et al.; Lan et al. and Dyer D H et al., The structural determination of an insect sterol carrier protein-2 with a ligand bound C16 fatty acid at 1.35 Å resolution. J Biol Chem 2003 278:39085-39091).

Several small molecules have been reported as inhibitors to the mosquito SCP-2, whereby the compounds are also reported to be lethal to both mosquitoes and tobacco hormworms likely due to a reduction in cholesterol uptake, and whereby the toxicity of N-(4-{[4-(3,4-dichlorophenyl)-1,3-thiazol-2-yl]amino}phenyl)acetamide in mice has been reported. (Kim M S et al., Identification of mosquito sterol carrier protein-2 inhibitors, J Lipid Res 2005 46(4):650-657). SCP-2 knockouts in mice have been reported to reduce the percentage of cholesterol absorbed in the intestine. (Fuchs M et al., Disruption of the sterol carrier protein 2 gene in mice impairs biliary lipid and hepatic cholesterol metabolism, J Biol Chem 276(51):48058-48065). It has also been reported that ethanol inhibits lipid binding to SCP-2. (Avdulov N A et al., Lipid binding to sterol carrier protein-2 is inhibited by ethanol, Biochimica et Biophysica Acta 1999 1437:37-45).

High serum cholesterol levels are linked to many medical conditions that threaten human health such as cardiovascular disease diabetics and Alzheimer's disease. (Martins I J et al., Apolipoprotein E, cholesterol metabolism, diabetes, and the convergence of risk factors for Alzheimer's disease and cardiovascular disease, Mol Psychiatry Jun. 20, 2006 and Michikawa M et al., The role of cholesterol in pathogenesis of Alzheimer's disease: dual metabolic interaction between amyloid beta-protein and cholesterol, Mol Nuerobiol 2003 27(1):1-12). It has also been reported that there exists a strong link between excess body weight and high cholesterol levels, which increases the health risk of people suffering from obesity. (Denke M A et al., Excess body weight: An under-recognized contributor to high blood cholesterol levels in white American men, Arch Intern Med 1993 153(9):1093-1103). Other reports suggest that the increased rate of obesity in the population necessitates an urgent need to manage cholesterol levels and reduce health risk. (Ogden C L et al., Prevalence of overweight and obesity in the United States 1999-2004, JAMA 2006 295(13):1549-1555 and Rutishauser J, The role of statins in clinical medicine-LDL—cholesterol lowering and beyond, Swiss Med Wkly 2006 136(3-4):41-49).

Mammals maintain serum cholesterol levels via two pathways: de novo biosynthesis mostly in liver tissue and absorption from food through the small intestine. The most commonly used cholesterol medicine are statins, which inhibit biosynthesis of cholesterol. (Stancu C et al., Statins: mechanism of action and effects, J Cell Mol Med 2001 5(4):378-387). Ezetimibe has also been reported to lower cholesterol by inhibiting absorption through the intestine. (Brown W V, Cholesterol absorption inhibitors: defining new options in lipid management, Clin Cardiol 2003 26(6):259-264). It has also been reported that a combination of a statin and an ezetimible synergistically lowers serum cholesterol levels. (Masana L et al., Long-term safety and tolerability profiles and lipid-modifying efficacy of ezetimibe coadministered with ongoing simvastatin treatment: a multicenter, randomized, double-blind, place-controlled, 48-week extension study, Clin Ther 2005 27(2):174-184).

SCP-2 over-expression enhances cholesterol uptake in both mammalian and mosquito cultured cells. (Moncecchi D et al., Sterol carrier protein-2 expression in mouse L-cell fibroblasts alters cholesterol uptake, Biochim Biophys Acta 1996 1302(2): 110-116).

### SUMMARY OF THE INVENTION

One aspect of the invention is a method for lowering serum total cholesterol concentration in a mammal by administering a therapeutically effective dose to a mammal (preferably a human) of a chemical compound that inhibits cholesterol and/or triglycerides from binding to the intracellular sterol carrier protein-2 (SCP-2). Such compounds are referred to as sterol carrier protein inhibitors (SCPIs). A particularly preferred SCPI is N-(4-{[4-(3,4-dichlorophenyl)-1,3-thiazol-2-yl]amino}phenyl)acetamide,

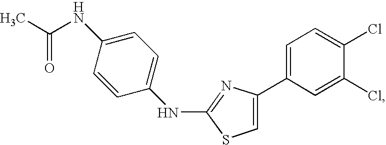

or a salt thereof. In an exemplary embodiment of the method, the salt is a hydrochloride salt or a hydrobromide salt.

Another aspect of the invention is a method for lowering serum low density lipoprotein cholesterol concentration in a mammal (preferably a human) by administering a therapeutically effective dose of N-(4-{[4-(3,4-dichlorophenyl)-1,3-thiazol-2-yl]amino}phenyl)acetamide,

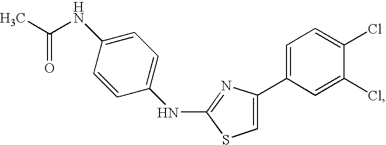

or a salt thereof, to the mammal. In an exemplary embodiment of the method, the salt is a hydrochloride salt or a hydrobromide salt.

Another aspect of the invention is a method for lowering serum triglyceride concentration in a mammal (preferably a human) by administering a therapeutically effective dose of N-(4-{[4-(3,4-dichlorophenyl)-1,3-thiazol-2-yl]amino}phenyl)acetamide,

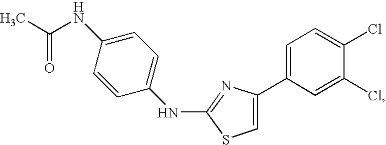

or a salt thereof, to the mammal. In an exemplary embodiment of the method, the salt is a hydrochloride salt or a hydrobromide salt.

### BRIEF DESCRIPTION OF THE DRAWINGS

FIG. 1 is a graph showing growth rates of young rats (starting at 20 days of age) upon oral administration of N-(4-{[4-(3,4-dichlorophenyl)-1,3-thiazol-2-yl]amino}phenyl)acetamide hydrobromide, whereby the effect on growth rate was insignificant, whereby the drug was administered 5 days/week at various dosages, whereby weight was measured every day during the treatment regimen (Monday-Friday), whereby the data is shown in three groups to compensate for variations in growth rate as the animals aged, whereby the listed days represent the number of days after the initial first administration, and whereby the bars represent the average growth rate±Standard deviation.

FIG. 2 is a graph showing total serum cholesterol levels in rats administered varying concentrations of N-(4-{[4-(3,4-dichlorophenyl)-1,3-thiazol-2-yl]amino}phenyl)acetamide hydrobromide for 5 days, whereby a significant decrease in cholesterol levels for all treated animals is demonstrated.

FIG. 3 is a graph showing serum ALT levels in rats administered various dosages of N-(4-{[4-(3,4-dichlorophenyl)-1,3-thiazol-2-yl]amino}phenyl)acetamide hydrobromide for 5 days, whereby no significant difference between the control (0 mg/kg) and the treated animals is demonstrated.

### DETAILED DESCRIPTION OF THE EXEMPLARY EMBODIMENTS

The instant invention is directed at the cholesterol and triglyceride lowering effects of sterol carrier protein-2 inhibitors (SCPIs) in mammals, such as humans. Rats were used to model the SCPIs therapeutic effect in a human. SCPIs are small molecular chemicals that, in theory, inhibit cholesterol and triglyceride serum levels by binding to the intracellular sterol carrier protein-2 (SCP-2). Even at high dosages, the instant SCPIs did not induce any acute toxicity in young rats as measured by growth rate, mortality and ALT levels.

N-(4-{[4-(3,4-dichlorophenyl)-1,3-thiazol-2-yl]amino}phenyl)acetamide and its preparation thereof is known in the art. (See ChemBridge Screening Library, Chembridge Corporation, San Diego, Calif., Registry No. 472980-58-8 (Jan. 12, 2005)). Preparation of salts thereof, such as hydrochloride and hydrobromide salts, is also well know in the art.

Therapeutically effective dosing for humans may be calculated using Allometric Scaling (AS) factors known in the art. For example, for a rat of 0.25 kg, the AS factor is around 4 assuming a human body weight of 70 kg.

### EXAMPLES

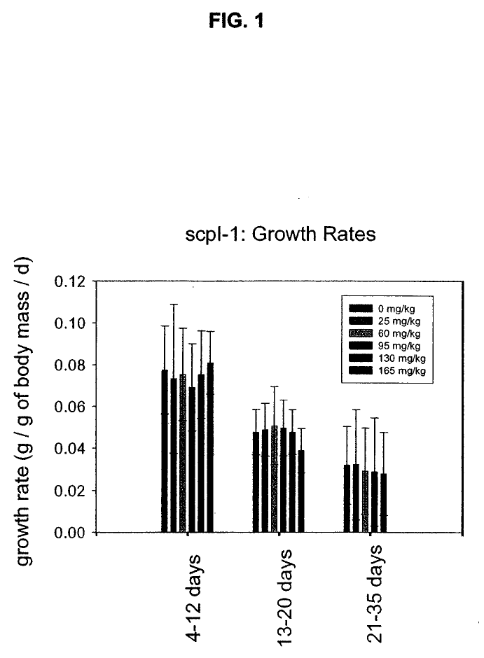

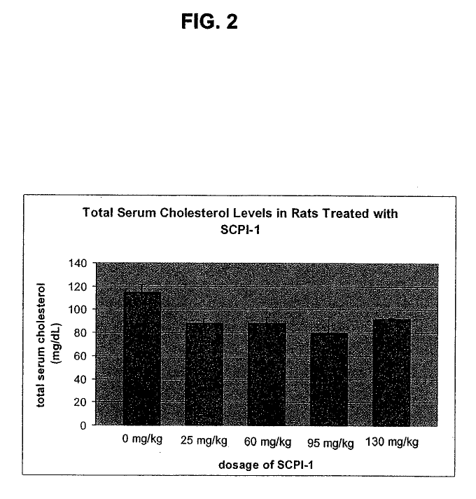

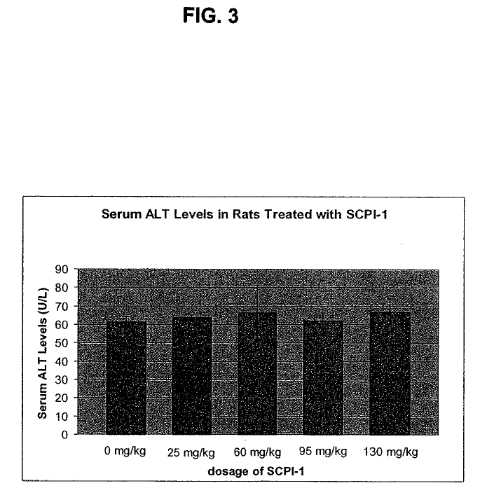

The data set forth in FIGS. 1-3 was generated in accordance with the following procedures.

Mammalian species (i.e., rats) were used. Rats were fed a standard diet (3.5% fat, <0.02% cholesterol). Weaned rats were grouped into treated and controls. Rats were chosen as the testing animal so that 0.5 ml of blood samples could be taken for testing. Rats were anesthetized first in an induction chamber with isoflurane and oxygen (100%). Once anesthetized rats were positioned dorsally for the blood draw from the jugular or ventral tail vein. An anesthetic nose-cone was placed on the nose of the rat allowing isoflurane and oxygen (100%) to be delivered through a Bain circuit. After obtaining blood, the rats were allowed to recover in a shoebox cage with a heat lamp positioned over the top of the cage. Once the rats were awake and moving freely around the recovery cage, they are placed back in their normal cages. Total time for procedure is ˜15-20 minutes per rat.

N-(4-{[4-(3,4-dichlorophenyl)-1,3-thiazol-2-yl]amino}phenyl)acetamide hydrobromide was mixed with DMSO (solvent) in increasing dosages, which were feed to laboratory rats. Rats received the preparations via oral drops. Daily observations were recorded concerning (i) weight changes and (ii) mortality. Blood samples were taken every three weeks for testing cholesterol levels and for testing Alanine Aminotransferase (ALT) for liver function. The blood chemistry tests were performed by the School of Veterinary Medicine, University of Wisconsin-Madison.

The vertebrate SCP-2 is involved in cholesterol metabolism, therefore, cholesterol homeostasis is advantageously monitored when SCP-2 inhibitors are tested in mammalian species. The major organ involved in cholesterol metabolism is the liver. ALT is an enzyme produced within the cells of the liver. The level of ALT increases under conditions where liver cells are inflamed or have undergone cell death. The level of ALT in tested animals was compared to the controls, whereby a significant elevation of ALT indicates that the tested compound impaires liver function. A 9 week evaluation was employed to assess acute toxicity of the compound.

Rats were given N-(4-{[4-(3,4-dichlorophenyl)-1,3-thiazol-2-yl]amino}phenyl)acetamide hydrobromide daily doses at 25 to 250 mg/kg body weight (the dosages were derived from the IC₅₀s in mouse cell cultures; Kim et al., 2005). The compound and controls (DMSO or water) were placed at the back of the mouth where they were immediately swallowed by the rat.

At the high dosages, the oral dosage form may be acutely toxic to rats. The animals were monitored on a daily basis for signs of distress such as inactivity, hunched posture, not eating or drinking, porphyrin staining around the eyes, and experiencing ≧10% dehydration.

I claim:

1. A method for lowering serum total cholesterol concentration in a human comprising administering a therapeutically effective dose of N-(4-{[4-(3,4-dichlorophenyl)-1,3-thiazol-2-yl]amino}phenyl)acetamide, 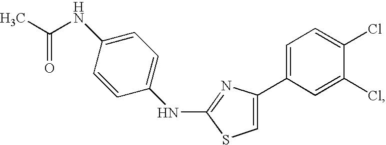
or a salt thereof, to the human.

2. A method for lowering serum low density lipoprotein cholesterol concentration in a human comprising administering a therapeutically effective dose of N-(4-{[4-(3,4-dichlorophenyl)-1,3-thiazol-2-yl]amino}phenyl)acetamide, 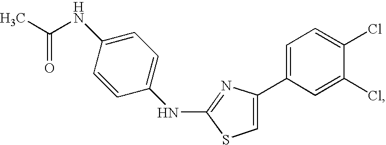
or a salt thereof, to the human.

3. A method for lowering serum triglyceride concentration in a human comprising administering a therapeutically effective dose of N-(4-{[4-(3,4-dichlorophenyl)-1,3-thiazol-2-yl]amino}phenyl)acetamide, 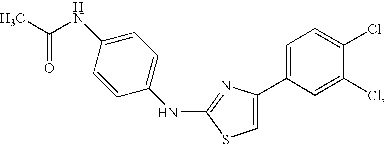
or a salt thereof, to the human.

4. The method of claims 1, 2 or 3, wherein the salt is a hydrochloride salt.

5. The method of claim 1, 2 or 3, wherein the salt is a hydrobromide salt.
